(19) United States
(12) Patent Application Publication
(10) Pub. No.: US 2023/0307147 A1
(43) Pub. Date: Sep. 28, 2023

(54) PATIENT HUB

(71) Applicant: Elmfield Partners, LLC d/b/a ViennaHealth, New York, NY (US)

(72) Inventors: Elizabeth McGuire, New York, NY (US); Norman Lavintman, New York, NY (US)

(21) Appl. No.: 18/125,379

(22) Filed: Mar. 23, 2023

Related U.S. Application Data
(60) Provisional application No. 63/323,389, filed on Mar. 24, 2022.

(51) Int. Cl. G16H 80/00; G16H 10/60

(52) U.S. Cl. CPC G16H 80/00; G16H 10/60

(57) ABSTRACT

A method includes receiving, by one or more processing circuits, a request to provision a plurality of entries associated with a patient; parsing, by the one or more processing circuits, a database to retrieve the plurality of entries in response to the request; identifying, by the one or more processing circuits, a source of the request; determining by the one or more processing circuits, a permission associated with the source of the request; parsing, by the one or more processing circuits, the plurality of entries based on the permission to retrieve one or more permitted entries of the plurality of entries that are accessible based on the permission; and displaying, by the one or more processing circuits, a note stream graphical user interface including a note stream providing the one or more permitted entries.

### CROSS-REFERENCE TO RELATED PATENT APPLICATION

This application claims the benefit of and priority to U.S. Provisional Patent Application No. 63/323,389, filed Mar. 24, 2022, which is incorporated herein by reference in its entirety.

### BACKGROUND

Various healthcare providers offer patient portals that allow patients to interact with and communicate with the providers. Such providers may include doctors, clinicians, and administrative staff. Patient portals accessible via a variety of electronic mediums may be useful for providing a central point of contact for communications regarding appointment scheduling, medical records, etc.

### SUMMARY

One embodiment relates to a method. The method includes receiving, by one or more processing circuits, a request to provision a plurality of entries associated with a patient; parsing, by the one or more processing circuits, a database to retrieve the plurality of entries in response to the request; identifying, by the one or more processing circuits, a source of the request; determining by the one or more processing circuits, a permission associated with the source of the request; parsing, by the one or more processing circuits, the plurality of entries based on the permission to retrieve one or more permitted entries of the plurality of entries that are accessible based on the permission; and displaying, by the one or more processing circuits, a note stream graphical user interface including a note stream providing the one or more permitted entries.

Another embodiment relates to a method. The method includes storing, by one or more processing circuits, a plurality of entries associated with a patient in a database; displaying, by the one or more processing circuits, a note stream graphical user interface with a note stream including at least a subset of the plurality of entries where the subset of the plurality of entries is determined based on a permission of a user accessing the note stream graphical user interface; arranging, by the one or more processing circuits, the subset of the plurality of entries in an order based on a timestamp associated with each entry; receiving, by the one or more processing circuits, a selection of a first entry of the subset of the plurality of entries by the user; and expanding, by the one or more processing circuits, the first entry into an expanded entry in response to a determination that a read receipt associated with a second entry preceding the first entry has been generated. The expanded entry includes additional information that is not visible prior to expansion of the first entry.

Still another embodiment relates to non-transitory computer-readable medium. The non-transitory computer readable medium has computer-executable instructions encoded therein. The instructions when executed by one or more processors cause the one or more processors to store a plurality of entries associated with a patient in a database, display a note stream graphical user interface with a note stream including at least a subset of the plurality of entries where the subset of the plurality of entries is determined based on a permission of a user accessing the note stream graphical user interface, arrange the subset of the plurality of entries in an order based on a timestamp associated with each entry, receive a selection of a first entry of the subset of the plurality of entries by the user, and expand the first entry into an expanded entry in response to a determination that a read receipt associated with a second entry preceding the first entry has been generated, wherein the expanded entry includes additional information that is not visible prior to expansion of the first entry.

This summary is illustrative only and is not intended to be in any way limiting. Other aspects, inventive features, and advantages of the devices or processes described herein will become apparent in the detailed description set forth herein, taken in conjunction with the accompanying figures, wherein like reference numerals refer to like elements.

### BRIEF DESCRIPTION OF THE FIGURES

FIG. 1 is a schematic diagram of a patient hub system, according to an exemplary embodiment.

FIG. 2 is a graphical user interface of a home screen shown to an administrative staff member, according to an exemplary embodiment.

FIG. 3 is a graphical user interface of a home screen shown to a clinician, according to an exemplary embodiment.

FIG. 4 is a graphical user interface of a home screen shown to a patient, according to an exemplary embodiment.

FIGS. 5, 6, and 7 are graphical user interfaces of an account screen shown to an administrative staff member, according to exemplary embodiments.

FIG. 8 is a graphical user interface of a form screen to be prepared by an administrative staff member, according to exemplary embodiments.

FIG. 9 is graphical user interface of a form summary screen shown to a clinician, according to an exemplary embodiments.

FIGS. 10 and 11 are graphical user interfaces of a form to be completed by a patient, according to an exemplary embodiment.

FIG. 12 is a graphical user interface of a patient listing screen shown to an administrative staff member, according to an exemplary embodiment.

FIG. 13 is a graphical user interface of a patient information screen shown to a clinician, according to an exemplary embodiment.

FIG. 14 is a graphical user interface of a staff listing screen shown to an administrative staff member, according to an exemplary embodiment.

FIG. 15 is a graphical user interface of a calendar screen shown to an administrative staff member, according to an exemplary embodiment.

FIGS. 16 and 17 are a graphical user interfaces of a calendar screen shown to various users, according to exemplary embodiments.

FIG. 18 is a graphical user interface of a settings screen shown to various users, according to an exemplary embodiment.

FIG. 19 is a graphical user interface of a medication details screen shown to a clinician, according to an exemplary embodiment.

FIG. 20 is a daily medication information screen shown to a patient, according to an exemplary embodiment.

FIG. 21 is a graphical user interface of a patient note screen shown to a clinician, according to an exemplary embodiment.

FIG. 22 is a graphical user interface of a patient note screen shown to a patient, according to an exemplary embodiment.

FIG. 23 is a graphical user interface of a patient note summary screen shown to a patient, according to an exemplary embodiment.

FIG. 24 is a graphical user interface of a message conversation overview screen shown to various users, according to an exemplary embodiment.

FIG. 25 is a graphical user interface of a message conversation screen shown to various users, according to an exemplary embodiment.

FIG. 26 is a graphical user interface of a note stream screen shown to various users, according to an exemplary embodiment.

FIG. 27 is a process for determining which notes to display for a particular user on a note stream, according to an exemplary embodiment.

### DETAILED DESCRIPTION

Referring generally to the Figures, systems and methods for an enhanced patient communications hub are disclosed according to various embodiments herein. In various embodiments, a computing device of a user (e.g., mobile device, smartphone, laptop computer, desktop computer, tablet, etc.) provides a patient hub that is associated with healthcare treatment pertaining to the user. As described in more detail below, a “patient hub” is a digital source of clinical information and communications provided on a user's device. The patient hub may include a schedule of appointments, payment portals, remote appointment portals, messaging portals, and a collection of treatment related notes (e.g., “note streams”). Note streams may include communications from clinicians (e.g., doctors, etc.) and/or other medical professionals (e.g., nurses, etc.), communications from patients, communications from healthcare administrative staff, and/or communications from parties associated with the patient (e.g., family members, caretakers, etc.). Additionally, the patient hub may include many other capabilities, functions, and features that are described below in more detail.

The systems and methods described herein improve upon a user's experience in managing clinical treatment by providing a dynamic stream of healthcare notes with tailored permission levels provided in an enhanced patient hub.

In an example illustrative scenario, a patient hub may include a note stream that generally features a series of treatment information, comments, or other notes regarding the patient. The note stream may limit access to particular notes based on the user's identity (and such limitations may be customizable by a user such as the patient or administrator), require users to view preceding unread notes before viewing and/or posting subsequent notes, and/or ensure that notes are displayed at or under a maximum character length to facilitate improved review by one or more users (e.g., in the form of a “tweet” format, a snippet, etc.), which may be selectively expandable to get a fuller or more in-depth version of a respective note.

Before turning to the Figures, which illustrate certain example embodiments in detail, it should be understood that the present disclosure is not limited to the details or methodology set forth in the description or illustrated in the figures. It should also be understood that the terminology used herein is for the purpose of description only and should not be regarded as limiting.

### System Overview

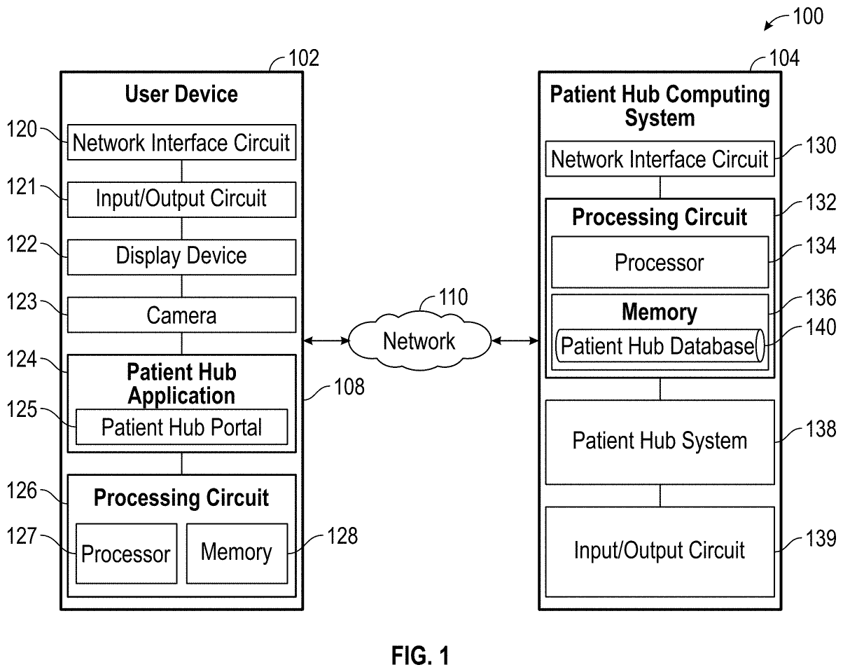

Referring now to FIG. 1, a system 100 for providing a dynamic and improved patient hub is shown, according to one embodiment. The system 100 includes a first device (e.g., a portable computing device, a stationary computing device, etc.), shown as user device 102, and a second device, server, or cloud computing system, shown as patient hub computing system 104. The patient hub computing system 104 includes a memory 136, which includes a patient hub database 140. The patient hub database 140 may store information regarding patients and treatments regarding the patients. In operation, a user may register, via the user device 102, a patient hub account with the patient hub computing system 104. As described in greater detail below, once the user has a patient hub account, the user may use the user device 102 to manage and/or access the information regarding one or more patients and the treatments thereof (operated by the patient hub computing system 104 and stored on the patient hub database 140) to varying degrees, depending on access and/or permission levels associated with the identity of the user.

In some embodiments, the user device 102 includes any type of device operated by a user in connection with services provided by a healthcare institution. As such, the user device 102 includes, but is not limited to, a phone (e.g., a smartphone), a portable computing device (e.g., a tablet computer, a laptop computer, a personal digital assistant, etc.), a wearable device (e.g., a smart watch, smart glasses, a smart bracelet, etc.), a desktop computer, and so on. The user device 102 may be owned by or otherwise associated with a user.

As used herein, the term “user” may refer to multiple different parties. The identify of such parties may affect the particular function of a patient hub application 124 stored on, accessed by, or operated by the user device 102, as described in greater detail below. While the patient hub application 124 may be described herein as being an application on the user device 102, in some embodiments, the patient hub application 124 is a website accessible using the user device 102 through the Internet (e.g., does not need to be locally stored on the user device 102). In some embodiments, the user is a patient. In other embodiments, the user is a clinician (e.g., a doctor, therapist, nurse, psychiatrist, surgeon, etc.). In other embodiments, the user is an administrative staff member of a healthcare institution. In other embodiments still, the user is a party associated with the patient (e.g., a family member, a spouse, a friend, a guardian, a caretaker, etc.). Various users may have access (to varying extents) to information regarding a particular patient. For example, where the user is the patient receiving treatment, the patient may have access to some or all of the information pertaining to the patient via the patient hub application 124. Further, other parties may register with the patient hub application 124 to have access to the patient's information. Further still, some parties (such as clinicians and administrative staff members) may have access to more information than a patient via the patient hub application 124. While other parties associated with the patient, such as family members, may have access to less information than the patient, the clinicians or other medical professionals, and the administrative staff. Thus, more than one user may have access to a respective patient's information at one time and, accordingly, more than one user may use a particular instance of the patient hub application 124. For example, a patient may use or have access to a first instance or version of the patient hub application 124, while a clinician may use or have access to a second instance or version of the patient hub application 124, and a family member of the patient may use or have access to yet a third instance or version of the patient hub application 124. Of course, each instance or version of the patient hub application 124 may be used on a distinct user device. In this sense, the system 100 may further include additional user devices similar to, or the same as, the user device 102 for each user associated with a patient's treatment or consultation.

As shown in FIG. 1, the user device 102 includes a network interface circuit 120, an input/output circuit 121, a display device 122, a camera 123, a processing circuit 126, and the patient hub application 124. The network interface circuit 120 may be used to establish connections via a network 110 (e.g., a wireless network, a wired network, etc.) between the user device 102 and the patient hub computing system 104, and/or other user devices in the system 100. The processing circuit 126 includes a processor 127, a memory 128, and is communicably coupled to the patient hub application 124.

The network interface circuit 120 may include one or more antennas or transceivers and associated communications hardware and logic (e.g., computer code, instructions, etc.). The network interface circuit 120 may include program logic that is structured to allow the user device 102 to access and couple/connect to the network 110 to, in turn, exchange information with, for example, the patient hub computing system 104, and/or other user devices in the system 100. That is, the network interface circuit 120 is coupled to the processor 127 and memory 128 and configured to enable communication with and through the network 110. The network interface circuit 120 may allow for the user device 102 to transmit and receive internet data and telecommunication data. Accordingly, the network interface circuit 120 may include any one or more of a cellular transceiver (e.g., CDMA, GSM, LTE, etc.), a wireless network transceiver (e.g., 802.11X, ZigBee, WI-FI, Internet, etc.), and a combination thereof (e.g., both a cellular transceiver). Thus, the network interface circuit 120 may enable connectivity to wireless area network (“WAN”) and/or as local area network (“LAN”) (e.g., via Bluetooth, NFC, Wi-Fi, etc. transceivers). Further, in some embodiments, the network interface circuit 120 includes cryptography capabilities to establish a secure or relatively secure communication session between other systems such as the patient hub computing system 104, and/or the other user devices in the system 100. In this regard, information (e.g., account information, login information, patient data, messages, and/or other types of data) may be encrypted and transmitted to prevent or substantially prevent a threat of hacking or other security breach.

In some embodiments, the input/output circuit 121 is structured to receive communications from and provide communications to a user of the user device 102. In this regard, the input/output circuit 121 is structured to exchange data, communications, instructions, etc. with an input/output component of the user device 102. The input/output circuit 121 may include hardware and associated logic (e.g., instructions, computer code, etc.) to enable the user device 102 to exchange information with a user and other devices (e.g., the patient hub computing system 104, other mobile devices, etc.) that may interact with the user device 102.

In some embodiments, the input aspect of the input/output circuit 121 allows the user to input or provide information into the user device 102 and may include machine-readable media for facilitating the exchange of information between the input/output device and the components of the user device 102. The input/output circuit 121 may include any combination of hardware components, for example, a mechanical keyboard, a touchscreen, a microphone, a camera (e.g., the camera 123), a fingerprint scanner, a device engageable to the user device 102 via a connection (e.g., USB, serial cable, Ethernet cable, etc.), and so on. The output aspect of the input/output circuit 121 allows the user to receive information from the user device 102, and may include, for example, a digital display, a speaker, illuminating icons, light emitting diodes (“LEDs”), and so on. The input/output circuit 121 may also include systems, components, devices, and apparatuses that serve both input and output functions. Such systems, components, devices and apparatuses may include, for example, radio frequency (“RF”) transceivers, near-field communication (“NEC”) transceivers, and other short range wireless transceivers (e.g., Bluetooth®, laser-based data transmitters, etc.). The input/output circuit 121 may include communication circuitry for facilitating the exchange of data, values, messages, and the like between an input/output device and the components of the user device 102. Accordingly, the input/output circuit 121 may also include other hardware, software, and firmware components that may otherwise be needed for the functioning of the user device 102.

In some embodiments, the display device 122 may be a screen, a touchscreen, and the like. The user device 102 may use the display device 122 to communicate information to the user (e.g., by displaying the information to the user on the display device 122) and/or to receive communications from the user (e.g., through a keyboard provided on a touchscreen of the display device 122). As described in greater detail below, the user device 102 may use the display device 122 to communicate a series or stream of messages or notes regarding the treatment of the patient. In some embodiments, the display device 122 is a component of the input/output circuit 121, as described above.

In some embodiments, the user device 102 may generate and/or receive and present various display screens that include information relating to a patient's treatment, including account information, treatment instructions, payment instructions, user messages, prescriptions, appointment schedules, appointment reminders, and so on. The various display screens may be accessed via the patient hub application 124. In some embodiments, a display screen may be used to request authentication information from the user. Authentication information may be used to unlock the user device 102 and/or open up and use the patient hub application 124. Thus, authentication information may include, but is not limited to, a credential (e.g., username and password information for the client application), biometric information (e.g., face scan, retina scan, fingerprint, etc.) for unlocking the user device 102 and accessing the patient hub application 124, and so on. Such display screens are presented to the user via the display device 122. As suggested above and described in further detail below, the various display screens generated by the user device 102 may vary depending on the identity of the user.

In some embodiments, the patient hub application 124 is structured to perform a variety of functions. The patient hub application 124 is communicably coupled via the input/output circuit 121 over the network 110 to the patient hub computing system 104 for performing one or more functions described herein, including providing a note stream as provided herein. In some embodiments, the patient hub application 124 is configured to interface with the patient hub computing system 104 to allow a user to manage the user's patient hub account. As suggested above, such management may vary depending on the user's identify. For example, where the user is a patient, management may include managing the patient's appointments (e.g., registering for one or more appointments), sending messages to the clinician, providing insurance information, payments, etc. As another example, where the user is a clinician, management may include managing the clinician's appointments with the patient (e.g., managing availability for the patient to schedule an appointment), viewing patient medical records, sending messages to the patient, sending prescriptions to the patient, posting treatment, test, or other notes, and so on. As another example, where the user is a family member of the patient, such management may include sending messages to the doctor or posting notes regarding the patient. As another example, where the user is an administrative staff member, such management may include viewing insurance information and receiving payments. It should be appreciated that any of such functions described herein may be applicable to any number of different users of the patient hub application 124.

In some embodiments, the patient hub application 124 allows the patient to attend a remote appointment via the camera 123 and a microphone of the user device 102. A doctor or clinician may use another user device in the system 100 to attend the remote appointment. Accordingly, in various arrangements, the patient hub application 124 is communicably coupled via the network interface circuit 120 to the patient hub computing system 104, and/or other user devices communicably coupled to the network 110 in the system 100.

In some embodiments, the patient hub application 124 may be structured to generate and present, control, and otherwise manage displays or graphical user interfaces on the user device 102. For example, the patient hub application 124 may enable the user to input information pertaining to completing the various functions and/or management described herein. The patient hub application 124 may communicate with the input/output circuit 121 and/or the display device 122 to perform such tasks.

In some embodiments, and as suggested above, the patient hub application 124 is structured to provide a user with access to a variety of activities regarding information associated with the patient. The user may interact with the patient hub application 124 via the display device 122 and the input/output circuit 121. In some arrangements, the user may only access the patient hub application 124 upon providing requisite authentication information, such as username and password information, biometric information, etc. In this way, the user's access to and use of the patient hub application 124 is conditioned upon the user's authentication, which may require the user to register for an account. In some embodiments, the user may be required to create a user client application account and/or register in order to access to the patient hub application 124. Upon receiving requisite authentication information, the patient hub application 124 may be structured to initiate an authenticated session, whereupon the user may have access to the functionality of the patient hub application 124. The “authenticated session” refers to a usage session of the patient hub application 124 following authentication of the user into the patient hub application 124. The authenticated session may expire after a certain duration of time with or without action of the user related to the patient hub application 124. The patient hub application 124 may be structured to prompt the user to provide authentication information in the event the authenticated session expires. As suggested above, multiple different “users” (clinician, patient, family, administrators, etc.) may be able to use the patient hub application 124 to access the variety of activities regarding information associated with a respective patient.

In some embodiments, the patient hub application 124 includes a circuit embodied within the user device 102. For example, the patient hub application 124 may include program logic stored in a system memory of the user device 102. In such arrangements, the program logic may configure a processor of the user device 102 to perform at least some of the functions discussed herein with respect to a patient hub system 138 of the patient hub computing system 104. In some embodiments, the patient hub application 124 is a web-based application, and many of the functionalities are provided by the patient hub system 138 of the patient hub computing system 104. Additionally, as will be understood, the level of functionality that resides on the user device 102 versus the patient hub computing system 104 will vary depending on the implementation.

As suggested above, in some embodiments, the patient hub application 124 is structured to enable the user to manage the information regarding a patient stored in the patient hub database 140. In this regard, the patient hub application 124 is structured to present, control, and otherwise manage displays or graphical user interfaces on the user device 102 including information related to the patient. For example, the patient hub application 124 may present the user with displays enabling the user to input information pertaining to scheduling appointments. The patient hub application 124 may then process the information input by the user, identify account information, and transmit the information to the patient hub computing system 104 for storage (e.g., in the patient hub database 140 in association with the user). The patient hub application 124 may allow the user to manually input information relating to the patient (e.g., a note, a question, etc.) and/or take a picture of an item (e.g., a picture of the user, a picture of a prescription, etc.). The patient hub application 124 may then process the input information relating to the item to be registered and transmit the information to the patient hub computing system 104 for storage.

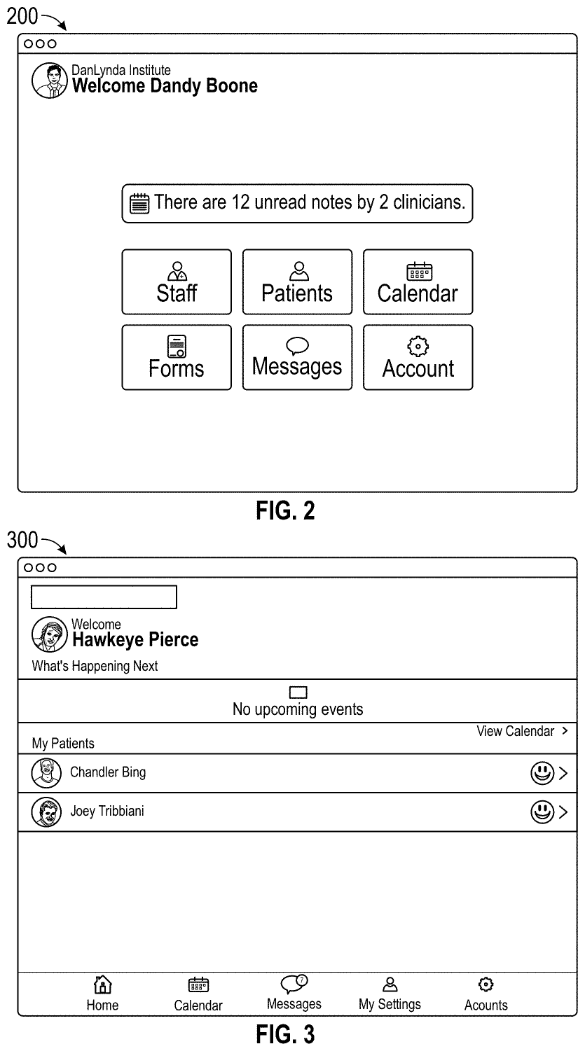

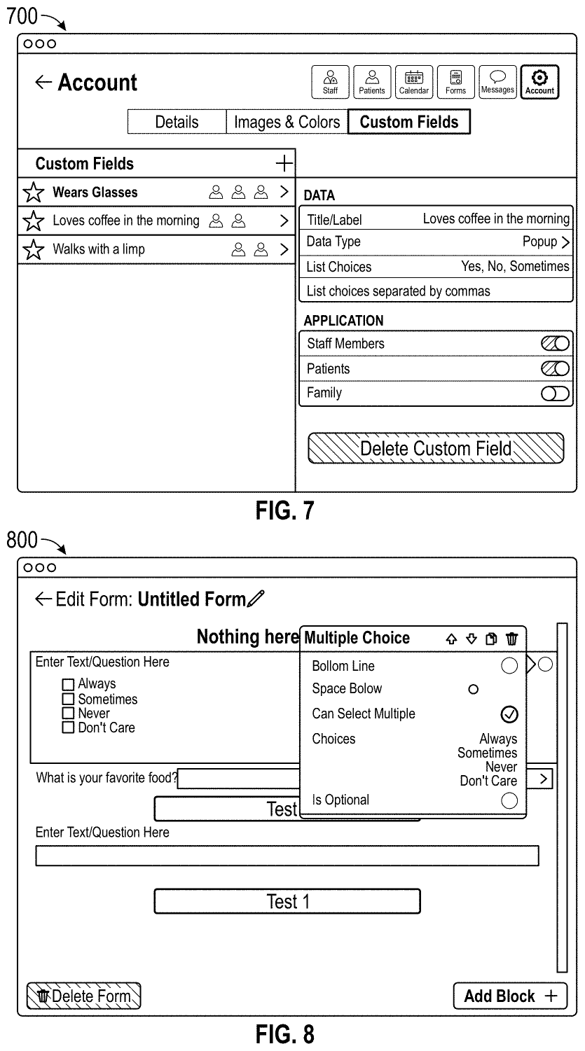

As shown in FIG. 1, the patient hub application 124 includes a patient hub portal 125. For example, the patient hub portal 125 may be accessed by the user while operating the patient hub application 124. The patient hub portal 125 may facilitate the user providing (e.g., uploading, inserting, copy/pasting, attaching, etc.) the patient hub computing system 104 with one or more pieces of information for storage in the patient hub database 140. The patient hub portal 125 may facilitate user retrieval (e.g., viewing) and/or editing one or more pieces of information stored in the patient hub database 140. As described in greater detail below, in some embodiments, the patient hub portal 125 is navigable via the display device 122. Navigation of the patient hub portal 125 via the display device 122 is described in further detail below in regards to FIGS. 2-26. It is to be understood that while the patient hub portal 125 has been shown as being part of the patient hub application 124, in other embodiments, the patient hub portal 125 may be a separate and stand-alone application.

In some embodiments, the patient hub computing system 104 is structured to permit, enable, facilitate, manage, process, and otherwise allow management of the patient hub application 124. In this regard, the patient hub computing system 104 may store information relating to the user device 102 and the operation of the patient hub application 124 and the patient hub portal 125 located therein. The patient hub computing system 104 may be associated with, owned by, and/or otherwise operated by a patient hub provider. In one embodiment, the patient hub provider is a healthcare institution. In another embodiment, the patient hub provider is a healthcare provider. In another embodiment still, the patient hub provider is a third party.

As shown in FIG. 1, the patient hub computing system 104 includes a network interface circuit 130, a processing circuit 132, and a patient hub system 138. The network interface circuit 130 includes hardware and/or program logic that facilitates connection of the patient hub computing system 104 to the network 110. Accordingly, the network interface circuit 130 supports communication between the patient hub computing system 104 and other components of the system 100 via the network 110, such as between the patient hub computing system 104, the user device 102, and other user devices on the network 110.

In some embodiments, the processing circuit 132 includes a processor 134 and a memory 136. The memory 136 includes the patient hub database 140. The patient hub database 140 is structured to retrievably store information regarding patient hub accounts held by various patients. The patient hub database may store the entirety of the information relating to a patient's treatment. However, as described in greater detail below, certain information stored on the patient hub database 140 may not be retrievable or accessible by one or more users of the patient hub application 124 based on an access level associated with the particular user. For example, the patient hub database 140 may store information related to the user and/or the user device 102, such as authentication information (e.g., username/password combinations, device authentication tokens, security question answers, etc.), upon registration of one or both for a patient hub.

In some embodiments, the patient hub system 138 is structured to provide patient hub services for the user, including providing a patient hub on the user device 102 and allowing the user to configure the patient hub. In some embodiments, the patient hub system 138 is structured to provide a patient hub client application (e.g., the patient hub application 124 discussed above) on the user device 102. In this regard, the patient hub system 138 enables registration of a user for a patient hub account, presents the user with various user interfaces (including the patient hub application 124 and the patient hub portal 125 included therein) enabling the user to use or manage the patient hub (e.g., view, add, remove, or otherwise modify information pertinent to the patient).

### Patient Hub Location Services

As suggested above, the network interface circuit 120 includes any one or more of a cellular transceiver (e.g., CDMA, GSM, LTE, etc.), a wireless network transceiver (e.g., 802.11X, ZigBee, WI-FI, Internet, etc.), and a combination thereof (e.g., both a cellular transceiver). In this sense, the user device 102 may be configured to transmit location data regarding the user (e.g., the patient hub application 124 may be configured to transmit location data to the patient hub computing system 104). Accordingly, various location alerts may be provided based on a user's location. For example, a patient with treatment pertaining to alcohol abuse may be provided with a patient hub application 124 that is configured to provide an alert to other devices on the network 110 when the patient hub application 124, via the location data collected by the network interface circuit 120, determines that the patient has been in a bar or other alcohol providing establishment for more than a threshold period of time (e.g., 5 minutes, 15 minutes, 30 minutes, 1 hour, etc.). While the above example relates to alcohol abuse, such location and time-based alerts may be applied to any particular treatment and/or monitoring of a patient (e.g., drug abuse, alcohol abuse, a court administered restraining order, an applied curfew, etc.).

### Patient Portal Graphical User Interfaces

Referring generally to FIGS. 2-26, and as suggested above, a user may navigate the patient hub application 124 and the patient hub portal 125 therein to interact with various display screens or graphical user interfaces (“GUIs”) that include information relating to a respective patient and/or the treatment of the respective patient. As suggested above, the “user” may be an individual of varying relation to the patient, including the patient themselves, a party associated with the patient, a clinician or other medical professional, or an administrative staff member. In some embodiments, and as suggested above, the user's access to and use of the patient hub application 124 may be conditioned upon the user's authentication. Such authentication requires the user to identify the patient (e.g., by a patient name, number, ID, or other identifying data) and identify the user's relationship to the patient. Thus, the user device 102 interacts with the patient hub computing system 104 based on, or in conjunction with, an identification of the patient and an identification of the user. As described in greater detail below, the various display screens that include information relating to the respective patient and the treatment thereof presented to the user may vary depending on the identity of the user.

Referring now to FIG. 2, a welcome screen 200 is shown, according to one embodiment. As shown, the welcome screen 200 has been generated with reference to an identification that the user is an administrative staff member and thus shown to the administrative staff member. The welcome screen 200 includes various buttons to view information pertaining to the administrative staff member.

Referring now to FIG. 3, a welcome screen 300 is shown, according to one embodiment. As shown, the welcome screen 300 has been generated with reference to an identification that the user is a clinician or other medical professional treating patients and thus shown to the clinician or other medical professional. The welcome screen 300 includes various buttons to view information pertaining to the clinician (e.g., calendar, patient profiles/content/notes, etc.).

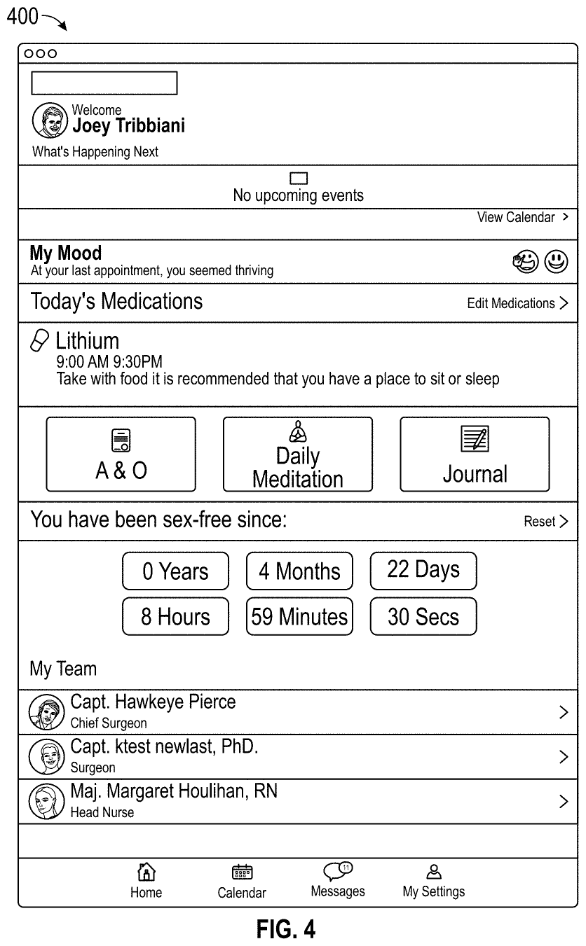

Referring now to FIG. 4, a welcome screen 400 is shown, according to one embodiment. As shown, the welcome screen 400 has been generated with reference to an identification that the user is a patient and thus shown to the patient. The welcome screen 400 includes various buttons to view and/or enter information pertaining to the patient (e.g., reminders regarding medications, a calendar, a mood entry interface, a list of team members or medical professionals associated with the patient's treatment, questions or polls pertaining to the patient's treatment, a journal, etc.).

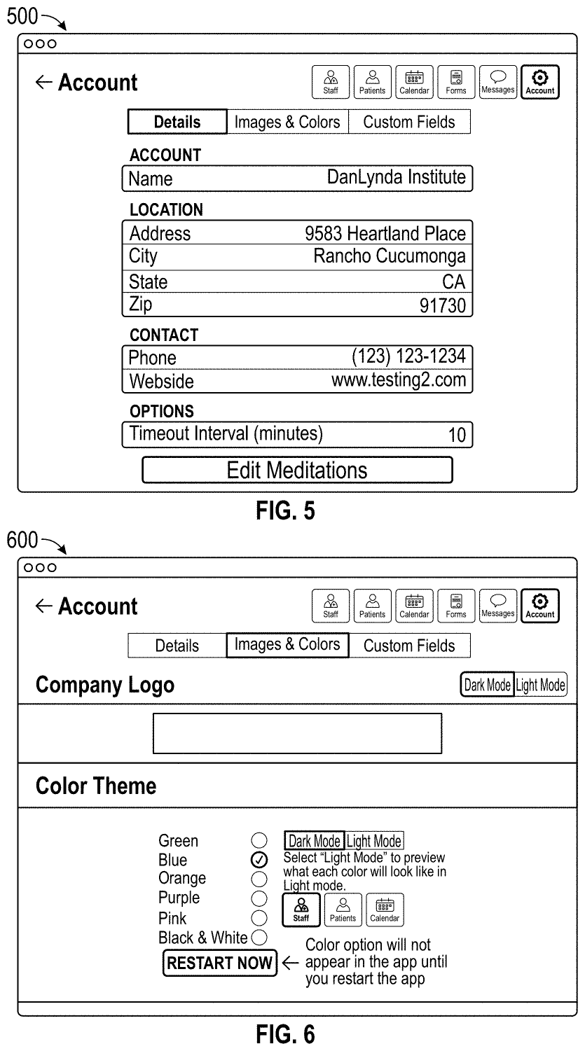

Referring now to FIGS. 5, 6, and 7, account screens 500, 600, and 700 are shown, according to some embodiments. The account screens 500, 600, and 700 may be used to generate and/or update account information regarding a user of the patient hub (e.g., name, address, contact information, application settings, entering custom personal information, providing access rights to profile or certain information associated with the profile, etc.).

Referring now to FIG. 8, a form generation screen 800 is shown, according to one embodiment. The form generation screen 800 may be shown to an administrative staff member and/or a clinician in order to prepare a form to provide to a patient for completion (e.g., via the patient hub application 124).

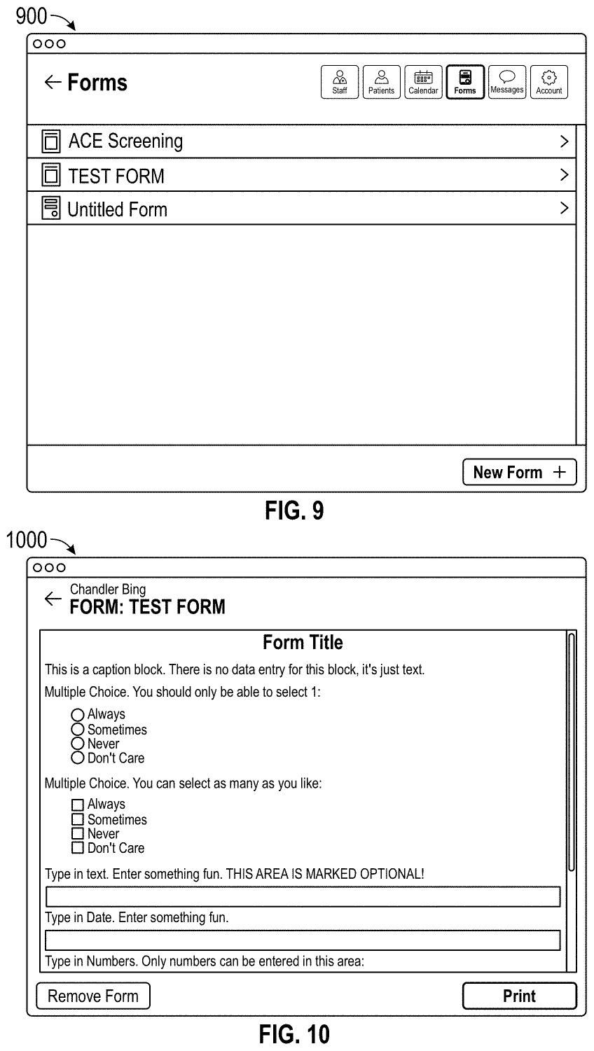

Referring now to FIG. 9, a form summary screen 900 is shown, according to one embodiment. The form summary screen 900 may be shown to a clinician to review one or more forms that may be provided to and/or that have been completed by a patient.

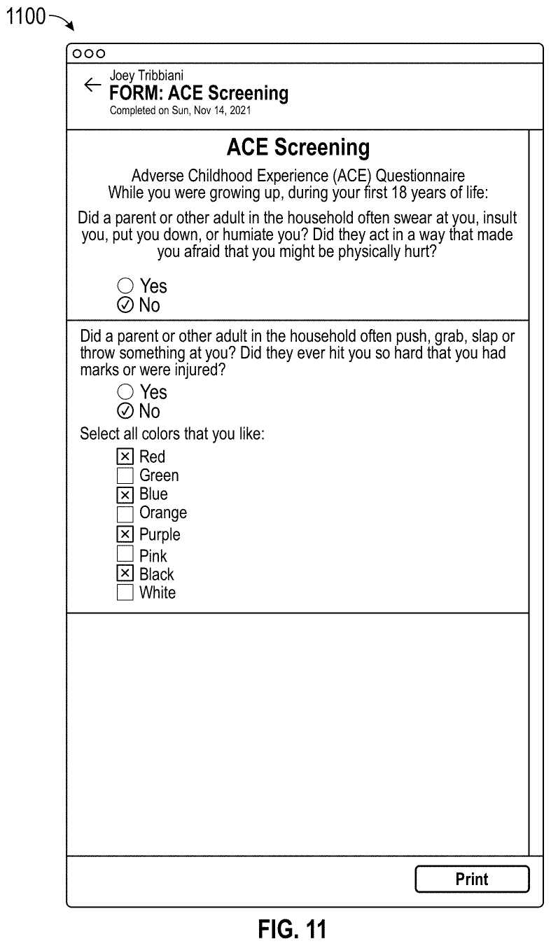

Referring now to FIGS. 10 and 11, a form screen 1000 and a form screen 1100 are shown, according to some embodiments. The form screens 1000 and 1100 may present forms (e.g., generated using the form generation screen 800) to be completed by a patient. As described in greater detail below in regards to FIG. 26, the patient's input submitted via the form screen 1000 and/or the form screen 1100 may be stored in the patient hub database 140 and reproduced in a note stream.

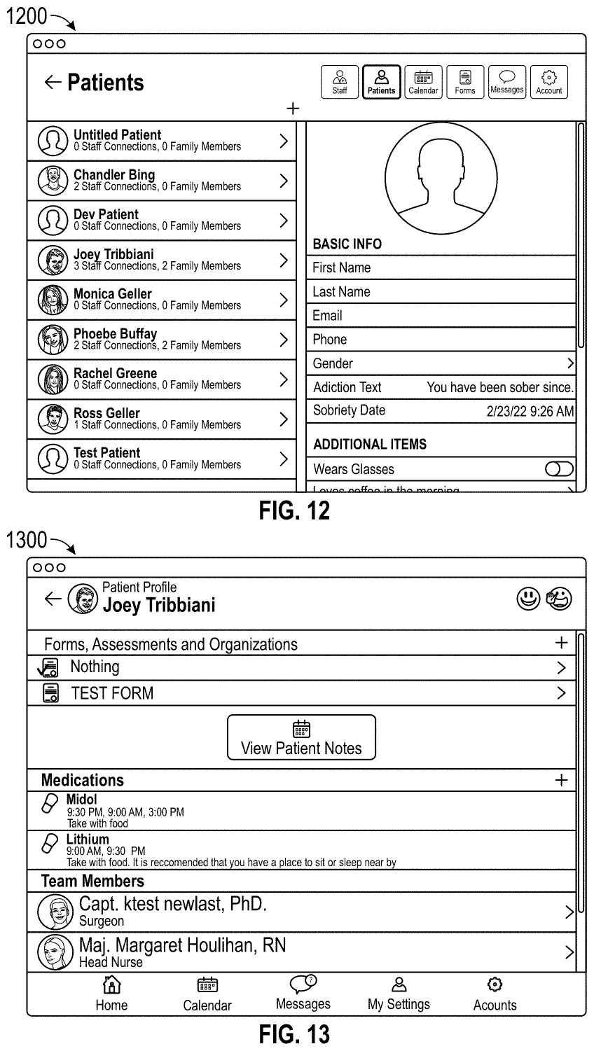

Referring now to FIG. 12, a patient listing screen 1200 is shown, according to one embodiment. The patient listing screen 1200 may be shown to a clinician or an administrative staff member and may provide a list of patients associated with the clinician or the administrative staff member.

Referring now to FIG. 13, a patient information screen 1300 is shown, according to an exemplary embodiment. The patient information screen 1300 may be shown to a clinician or an administrative staff member to view a summary of information pertaining to a patient (e.g., whether forms have been completed, what medications the patient is on, which other medical professionals are treating the patient, view a patient note steam, etc.).

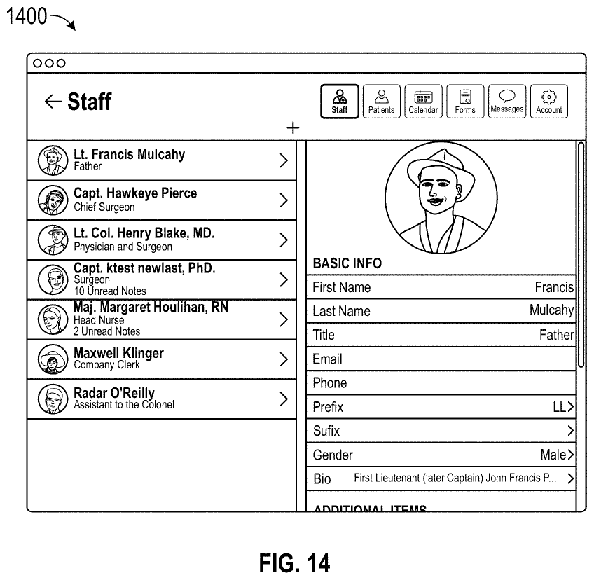

Referring now to FIG. 14, a staff listing screen 1400 is shown, according to one embodiment. The staff listing screen 1400 may be shown to an administrative staff member or a clinician to summarize one or more personnel involved in the treatment of a patient or generally employed by a healthcare facility.

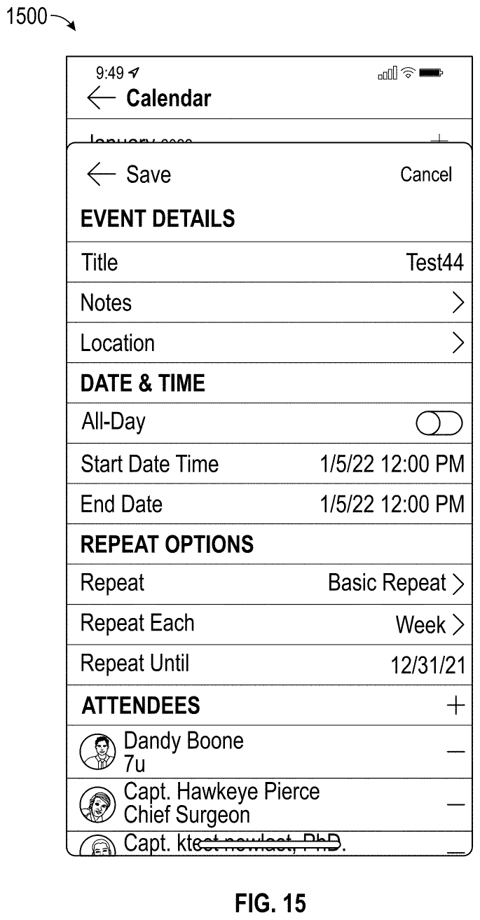

Referring now to FIG. 15, an appointment screen 1500 is shown, according to one embodiment. The appointment screen 1500 may allow a user to provide various inputs relating to the timing of an appointment, notes regarding the appointment, and a location of the appointment. As described in greater detail below in regards to FIG. 26, information accepted from a user via the appointment screen 1500 may be stored in the patient hub database 140 and reproduced on a note stream. In some embodiments, the various appointments may involve multiple parties. In other embodiments, one party (e.g., a therapy concierge) has exclusive permission to manage various user's calendar information as displayed by the appointment screen 1500. In some embodiments, a notification may be provided to alert a user of an upcoming appointment. For example, one or more popup messages may be displayed to the user via the patient hub application 124. As another example, the patient hub application 124 may access the input/output circuit 121 of the user device 102 to provide an audio and/or visual notification via the display device 122 regardless of whether the user is presently operating the patient hub application 124. It should be understood that in various embodiments disclosed herein regarding notifications regarding appointments or other treatment information, the patient hub application 124 may be configured to screen or scrub such notifications for sensitive patient information.

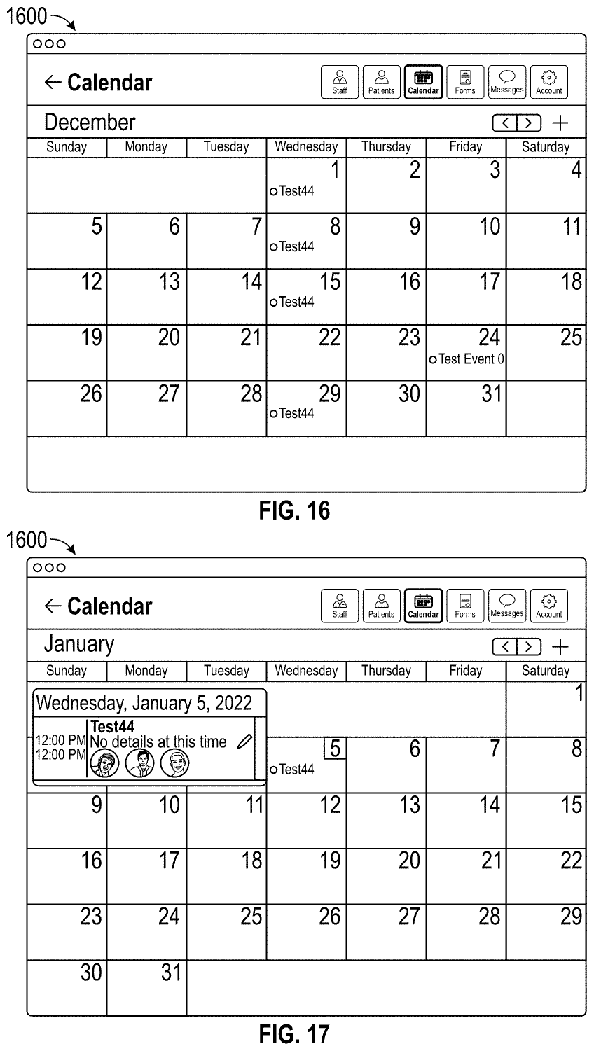

Referring now to FIGS. 16 and 17 a calendar screen 1600 is shown, according to some embodiments. The calendar screen 1600 may include time-related information regarding appointments, testing, and other activities to be performed by or with the patient.

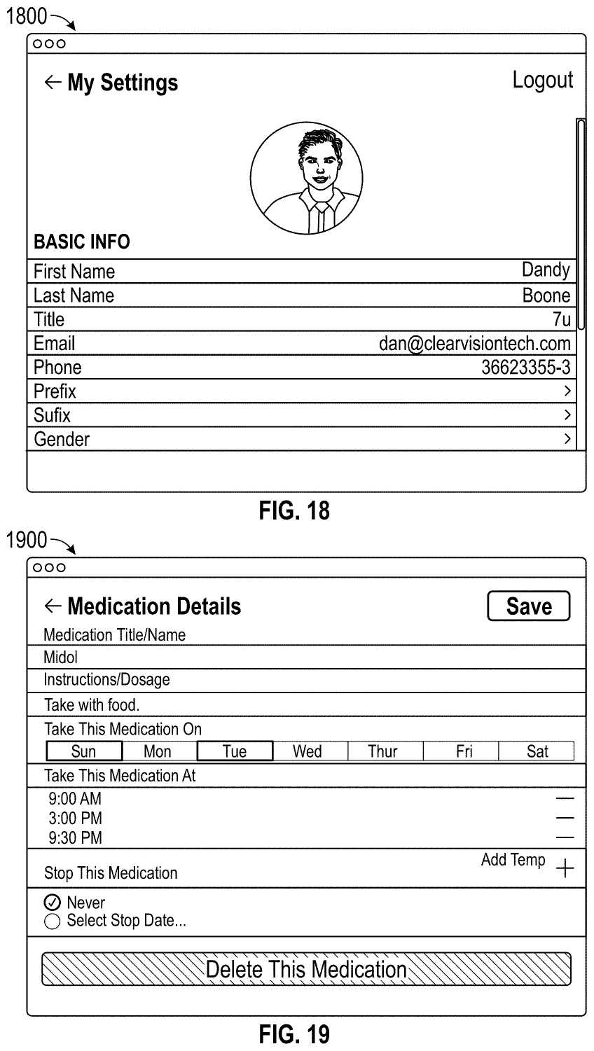

Referring now to FIG. 18, a settings screen 1800 is shown, according to one embodiment. The settings screen 1800 may allow a user to update contact and personal information pertaining to the user's use of the patient hub application 124.

Referring now to FIG. 19, a medication details screen 1900 is shown, according to one embodiment. The medication details screen 1900 may be shown to a clinician or other healthcare professional. The medication details screen 1900 may allow a clinician to prescribe one or more medications or treatments to be taken or performed on a given day and time. Further, the medication details screen 1900 may allow a clinician to provide a title for the medication, instructions and dosage information, and a duration of the treatment. As described in greater detail below in regards to FIG. 26, the information submitted via the medication details screen 1900 may be stored in the patient hub database 140 and reproduced in a note stream.

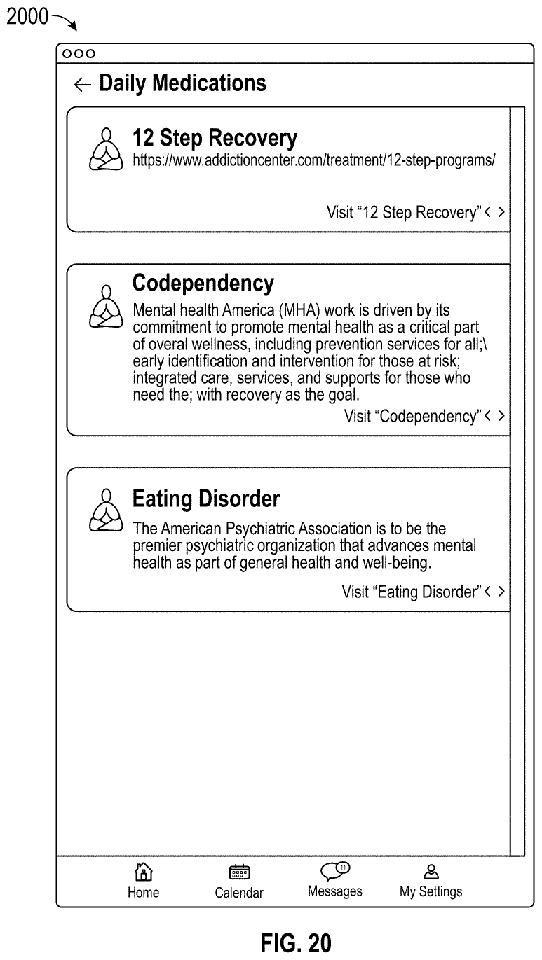

Referring now to FIG. 20, a daily meditation information screen 2000 is shown, according to one embodiment. The daily meditation information screen 2000 may be shown to a patient. The daily meditation information screen 2000 may include one or more selectable tiles that correspond to informational pamphlets, articles, or websites that a clinician suggests to a patient to help the patient along their treatment.

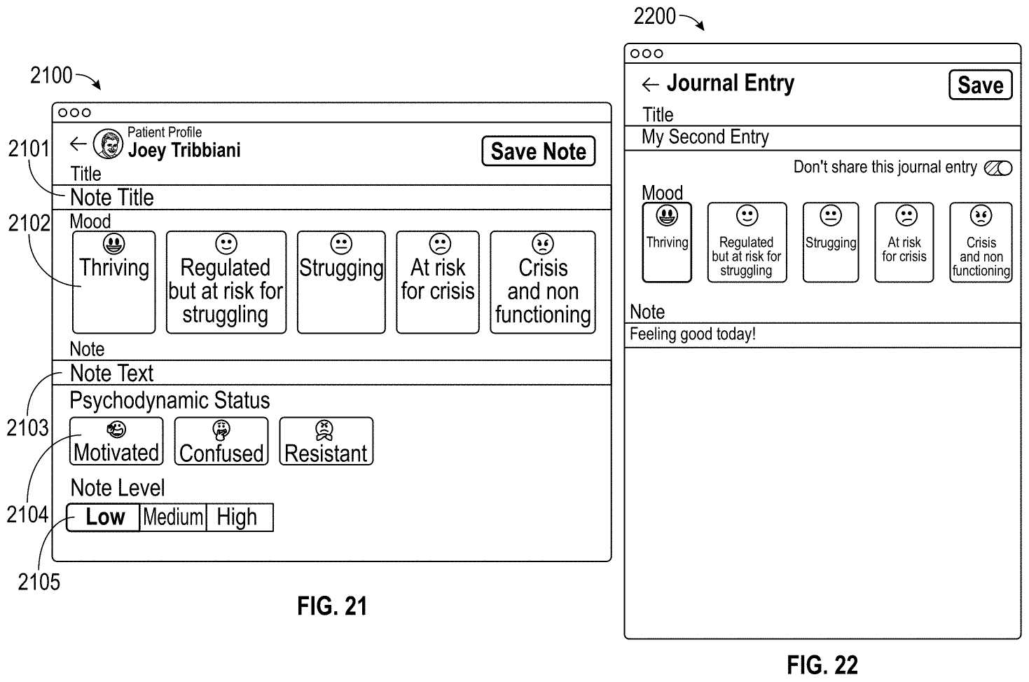

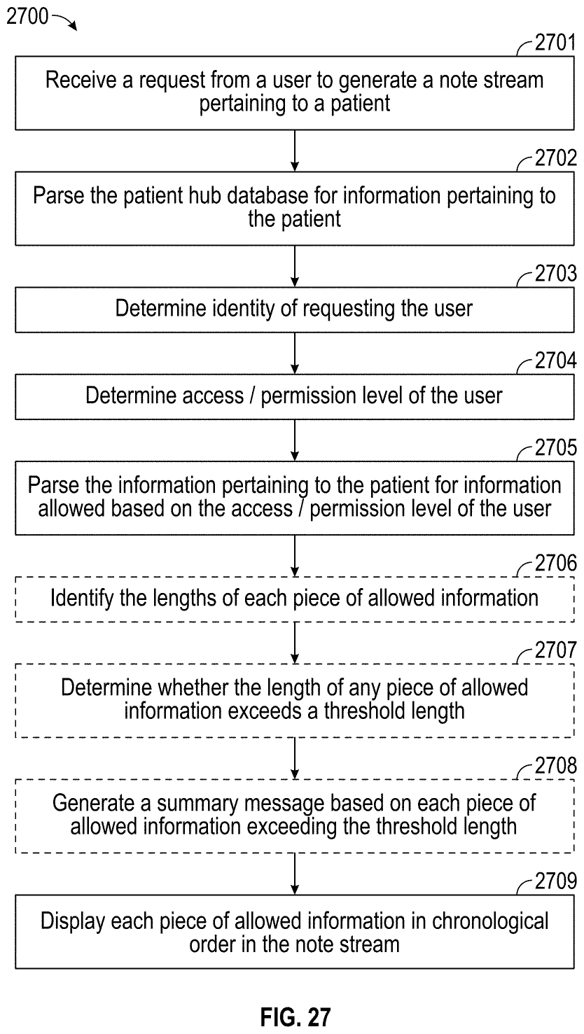

Referring now to FIG. 21, a note entry screen 2100 is shown, according to one embodiment. As shown, the note entry screen 2100 is formatted for clinician use. Though the note entry screen 2100 may be similarly used by a patient or third party associated with the patient (e.g., a family member, etc.). The note entry screen 2100 includes a title input 2101, a mood indicator selection 2102 providing a plurality of selectable mood options (e.g., thriving, regulated but at risk for struggling, struggling, at risk for crisis, crisis and nonfunctioning, etc.), a note input 2103, a psychodynamic status selection 2104 providing a plurality of status options (e.g., motivated, conflicted, resistant, etc.), and a note urgency level selection 2105 providing a plurality of urgency or importance level options (e.g., low, medium, high, etc.). The note entry screen 2100 may be used by a clinician to evaluate a patient during or following an appointment. In some embodiments, when an input is provided to either the title input 2101 and/or the note input 2103, the note may be sent from the user device 102 and received by the patient hub computing system 104 for review. The patient hub computing system 104, via the patient hub system 138, may compare the title input 2101 and/or the note input 2103 against a threshold length for desirable display (e.g., a “tweet length”) in a note stream page described in greater detail below in regards to FIG. 26. If the title input 2101 and/or the note input 2103 are of a character length that exceeds the threshold tweet length, the user may be prompted with one or more pop-up notifications to revise the title input 2101 and/or the note input 2103 to be of a shorter character length. Once the user has revised the title input 2101 and/or the note input 2103 to a sufficient length, the patient hub system 138 may store the note in the patient hub database 140. In other embodiments, the patient hub system 138 stores the note in the patient hub database 140 regardless, and the language of the note may be automatically altered to be of a desirable length as described in greater detail below in regards to FIG. 27. Ultimately, the note may be stored in the patient hub database 140 with an identification of the pertinent patient and the user submitting the note. The note may be stored in the patient hub database along with associated selections made in regards to the mood indicator selection 2102, the psychodynamic status indicator selection 2104, and the note urgency level selection 2105.

Referring now to FIG. 22, a journal entry screen 2200 is shown, according to one embodiment. As shown, the journal entry screen 2200 is formatted for patient use. In some embodiments, a note submitted by a patient may be referred to as a “journal entry.” The function of receiving and processing inputs via the journal entry screen 2200 may be similar to that of the note entry screen 2100 described in regards to FIG. 21. The journal entry screen 2220 may provide the patient with the option to share the journal entry (e.g., post the journal entry as a note to the note stream for clinician viewing) or not share the journal entry such that the journal entry remains private and only viewable by the patient (e.g., via a journal summary interface).

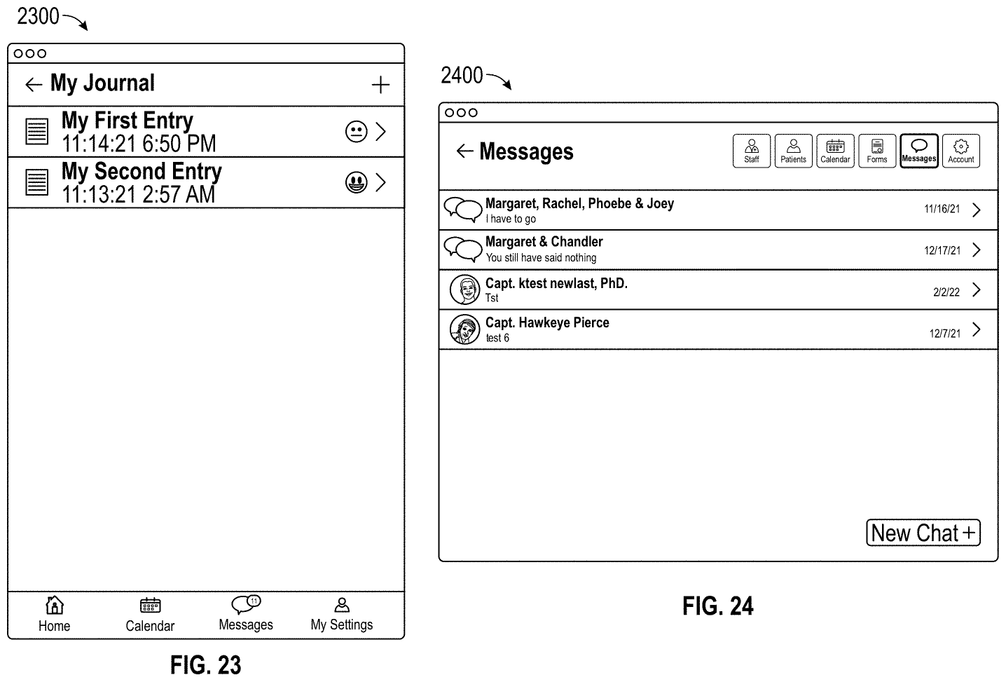

Referring now to FIG. 23, a journal summary screen 2300 is shown, according to one embodiment. As shown, the journal summary screen 2300 is formatted for patient use and may provide a patient journal summary including the journal entries crafted by the patient via the journal entry screen 2200. In this way, a patient may review one or more previous journal entries or select to add a new journal entry, which will lead the patient to the journal entry screen 2200.

Referring now to FIG. 24, a message conversation overview screen 2400 is shown, according to one embodiment. As shown, the message conversation overview screen 2400 includes one or more conversations between various users registered with the patient hub. Therefore, the particular user accessing the message conversation overview screen 2400 may communicate with various users of the patient hub. For example, two or more clinicians for a respective patient may be able to communicate directly with each other, a respective clinician may be able to communicate directly with a respective patient, a respective clinician may be able to communicate directly with a third party associated with the respective patient (e.g., a family member, a guardian, etc.), etc.

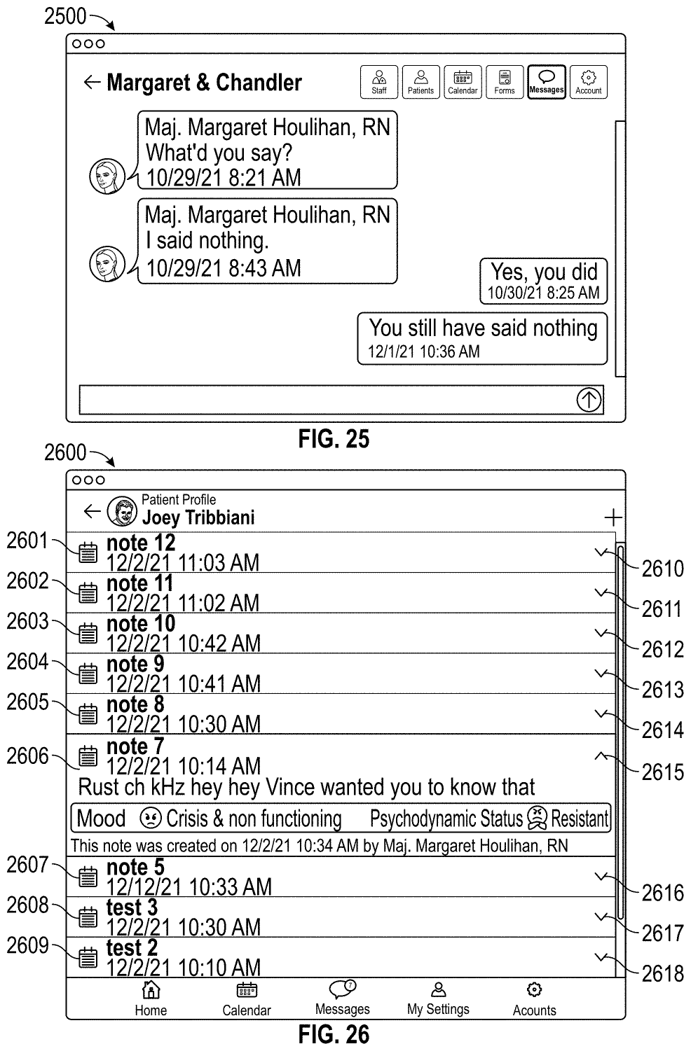

Referring now to FIG. 25, an individual conversation screen 2500 is shown, according to one embodiment. In some embodiments, the individual conversation screen 2500 may be generated based on a selection of one of the conversations displayed via the message conversation overview screen 2400. As shown, the individual conversation screen 2500 may be configured to receive a user input to send a direct message to other participants in the individual conversation.

In some embodiments, a patient may communicate with a clinician directly via the conversation screen 2500. In other embodiments, the patient may communicate with multiple clinicians via the conversation screen 2500. In other embodiments still, a patient may communicate with a clinician and family members in a group via the conversation screen 2500. In some embodiments, such conversations are monitored by a third party concierge.

In some embodiments, the various conversations via the conversation screen 2500 (or other communication interfaces described herein) are stored on the patient hub database 140 for a threshold period of time to comply with the Health Insurance Portability and Accountability Act (“HIPAA”).

Referring now to FIG. 26, with additional reference to FIG. 1, a note stream interface 2600 is shown, according to one embodiment. In general, the note stream interface 2600 displays some or all of a series or stream of notes or entries pertaining to a respective patient and the treatment thereof. A user may navigate the patient hub portal 125 within the patient hub application 124 and make a selection to view a note stream pertaining to the patient, such as the note stream presented via the note stream interface 2600.

In some embodiments, the contents of the note stream interface 2600 (e.g., the individual notes or entries) may be populated by information stored in the memory 128 and/or the patient hub database 140. In the case that the note stream interface 2600 is populated by information stored in the patient hub database 140, such information is transmitted via the network 110 as described above. As shown in FIG. 26, the note stream interface 2600 provides a note stream including a plurality of entries 2601-2609. Each of the entries 2601-2609 may pertain to treatment of the patient. At the same time, each of the entries 2601-2609 may be submitted from a variety of parties. For example, one or more of the entries 2601-2609 may be submitted by a clinician, a nurse, an administrative staff member, the patient, and/or a party associated with the patient (e.g., a family member, a friend, a guardian, etc.). In this sense, various users may access distinct instances of the patient hub application 124 (e.g., registered to each user) and navigate (via the patient hub portal 125) to a note or journal entry screen, such as the note entry screen 2100 and/or the journal entry screen 2200, to submit a note or entry to the note stream. It should be appreciated, however, that other user interfaces described herein may allow for user input such that a note or entry may be generated therefrom to populate the note stream interface 2600. As shown, each of the entries 2601-2609 are displayed in chronological or reverse chronological order (e.g., based on a timestamp associated with their submission to the patient hub).

In some embodiments, each of the entries 2601-2609 are displayed on the note stream interface 2600 in a form that is short enough to be readable over a condensed graphical space (e.g., one line of text, 20 characters or less in total, “tweet form,” etc.). The manual generation of notes or entries in “tweet form” is described in greater detail above in regards to FIG. 21 (e.g., may be based on the title entered via the title input 2101). Alternatively, the automatic generation of notes or entries in “tweet form” is described in greater detail below in regards to FIG. 27. A user may press one of the expansion keys 2610-2618 to expand a note or entry to reveal other information associated with the note or entry (e.g., may be based on the note or entry entered via the note input 2103, the longer note if originally provided in a form longer than “tweet” form, selections and identifications provided with the note or entry, an identification of the provider, poster, or submitter of the note or entry, etc.). As shown in FIG. 26, the entry 2606 has been expanded by selecting the expansion key 2615 to show additional information associated with the entry 2606. The additional information may includes a longer note narrative, a form for completion by the patient, a form completed by the patient, test results associated with the patient, prescribed medication details, a patient mood indicator, a patient psychodynamic indicator, and/or an authorship or note creator indicator.

In some embodiments, and as suggested above, the notes or entries populating the note stream interface 2600 may include, but are not limited to, messages/notes regarding a patient's treatment, test forms pertaining to a patient's treatment, prescriptions pertaining to a patients treatment, calendar notifications pertaining to a users's treatment (e.g., appointments), and/or any other pieces of information relayed among the various users submitting and receiving information pertaining to the patient. For example, a user such as an administrative staff member or a clinician may submit a form for a different user, such as the patient, to complete, as shown in FIGS. 8 and 9. In some embodiments, the forms submitted for completion via the form generation screen 800 (by a clinician or administrative staff member) may be extracted from the patient hub database 140 and be reproduced in the note stream interface 2600 and shown as an uncompleted test or form (as shown in the entry 2609). In other embodiments, a form completed by a patient, such as the form screens depicted in FIGS. 10 and 11, may be extracted from the patient hub database 140 and reproduced in the note stream interface 2600 and shown as a completed test or form (as shown in the entry 2608). As another example, upcoming calendar notifications, described in regards to FIGS. 15-17, may be extracted from the patient hub database 140 and reproduced in the note stream interface 2600. As another example still, medication information, described in regards to FIGS. 19 and 20, may be extracted from the patient hub database 140 and reproduced in the note stream interface 2600. As another example, clinician notes or entries, described in regards to FIGS. 21, may be extracted from the patient hub database 140 and reproduced in the note stream interface 2600, such as any of entries 2601-2609. As another example, patient journal notes, described in regards to FIGS. 22 and 23, may be extracted from the patient hub database 140 and reproduced in the note stream interface 2600, such as any of entries 2601-2609. As yet another example, messages sent between users pertaining to the patient, described in regards to FIGS. 24 and 25, may be extracted from the patient hub database 140 and reproduced in the note stream interface 2600 as any of entries 2601-2609.

In some embodiments, and as discussed herein, the note stream may be generated differently depending on the identity (and resulting access/permission levels) of the user accessing the note stream interface 2600. For example, a user associated with the patient (e.g., a family member) may be able to post notes or entries to the note stream for other users to view (e.g., the clinician, the patient, the administrative staff member). However, the family member may not be able to view or access the other notes or entries posted to the note stream. In this sense, the family member would only be able to view or access the notes or entries that the family member themselves posted to the note stream via the note stream interface 2600. Conversely, a clinician may be able to post notes or entries to the note stream and view or access all notes or entries posted to the note stream that are associated with a patient that the clinician is treating. It should be appreciated that various arrangements of access/permission levels may be applied to different categories of users depending on the particular patient or other relevant circumstances. For example, particular users may not be able to see notes or entries on the note stream interface 2600 that pertain to private or sensitive events in the patient's treatment. Such configurations may be set as a default or voluntarily set by the patient or clinician. The particular process of generating the note stream as it pertains to various viewing users is described in greater detail below in regards to FIG. 27.

In some embodiments, various levels of access/permission associated with the various users may be a default configuration. In other embodiments, the patient associated with the patient hub may voluntarily dictate the access/permission levels pertaining to one or more other users. For example, the patient may strip a clinician of access/permission to view various categories of notes or entries on the note stream interface 2600 (see, e.g., the “Don't share this journal entry” toggle on the journal entry screen 2200). In such a case where the patient updates one or more access/permission levels associated with a user, the various instances of the patient hub application for each user may be configured to provide a notice that there has been a change in access/permission levels regarding the note stream.

In some embodiments, a user may not be able to access a particular note or entry (e.g., by expanding the note or entry via one of the expansion keys 2610-2618) until the particular user has read every preceding note accessible to the user on the note stream interface 2600. In this sense, the user device 102 may provide the patient hub computing system 104 with a “read receipt” for each note or entry on the note stream interface 2600 when the user expands the note via one of the expansion keys 2610-2618. For example, the note stream interface 2600 is shown with entry 2606 expanded. Thus, the user accessing the note stream interface 2600 viewed the entries 2607-2609 prior to attempting to view the entry 2606. After viewing the entry 2606, the user may not view any of the entries 2601-2604 before viewing the entry 2605, and so on. Accordingly, users may be prevented from providing overlapping or irrelevant notes or entries to the note stream (in light of notes that the user otherwise would not have read) or this may ensure that the user has a full and complete understanding of the patient's file before proceeding with submitting new notes or entries, or reading newer notes or entries that may need the context of earlier submitted notes or entries.

Referring now to FIG. 27, with additional reference to FIGS. 1 and 26, a process 2700 for populating a note stream (such as the note stream shown in the note stream interface 2600 depicted in FIG. 26) is shown, according to one embodiment. At step 2701, a request is received from a user to generate a note stream pertaining to a patient. For example, a user may use the user device 102 to access the patient hub application 124 and the patient hub portal 125 therein. The user may then navigate the various interfaces described herein to open the note stream interface 2600. In some embodiments, opening the note stream interface 2600 may generate the user's request to generate the note stream pertaining to a respective patient. The request may be transmitted from the user device 102 (via the network interface circuit 120) over the network 110 to the patient hub computing system 104 (via the network interface circuit 130). Upon receipt of the request, the patient hub system 138 may evaluate the request. In some arrangements, the patient hub system 138 begins by identifying the patient associated with the request.

In some embodiments, at step 2702, the patient hub database 140 is parsed for information pertaining to the patient. For example, the patient hub database 140 may be arranged such that each piece of information stored in the database is tagged with a particular patient identifier. The information stored in the patient hub database 140 is scanned for information associated with the patient associated with the request. The matching information is then identified and set aside as information pertaining to the patient.

In some embodiments, at step 2703, the identity of the requesting user is determined. For example, the patient hub system 138 may receive authorization information regarding the user operating the user device 102 that sent the request.

In some embodiments, at step 2704, an access/permission level of the requesting user is determined based on the identity of the user. For example, the patient hub database 140 may store an identification of each user with an associated access/permission level in regards to various patients. The patient hub system 138 may identify the user from patient hub database 140 and determine the relevant access/permission level (e.g., clinician level permission, patient level permission, family level permission, admin level permission, etc.).

In some embodiments, at step 2705, the information set aside as pertaining to the patient (at step 2702) is parsed a second time based on the user identity. The information pertaining to the patient that the user is allowed to view based on the access/permission level associated with the user is extracted and once again set aside as information that the user may view in the note stream.

In some embodiments, steps 2706-2708 described next are omitted. In some embodiments, at step 2706, lengths of each piece of information that the user may view in the note stream is determined. For example, the patient hub system 138 may examine each of the piece of information that the user may view in the note stream and determine a character count for each piece of information.

In some embodiments, at step 2707, the patient hub system 138 determines whether any of the pieces of information that the user may view in the note stream have a length that exceeds a threshold amount of characters. For example, the patient hub system 138 may set the threshold amount as a number of characters that may fit in a single subject line regarding the piece of information on the screen of the user device 102 (e.g., the display device 122). Accordingly, the patient hub system 138 may identify each piece of information with an excessive character length for further processing before being displayed on the note stream.

In some embodiments, at step 2708, a summary message is generated based on each piece of information that the user is allowed to view in the note stream with an excessive character length as determined in the step 2707. In some arrangements, a summary message may be generated by removing semantically unnecessary characters from a piece of information (e.g., punctuation, leading spaces, trailing spaces, etc.). In other arrangements, the patient hub system 138 may access a thesaurus to determine words of similar meanings with shorter characters lengths to replace one or more words in the piece of information. In other arrangements still, the patient hub system may apply natural language processing techniques via a machine learning model to generate a consolidated summary of the piece of information. Here, the machine learning model may be generated by the patient hub system 138 with training data including other pieces of semantic data existing in the patient hub database 140 with shorter character lengths. Alternatively, as described above in regards to FIG. 26, the patient hub portal 125 may request that a user revise a message into a shorter form prior to submission or posting. The revision process (e.g., the long message and the short message) may be identified by the patient hub system 138 as training data for the machine learning model.

In some embodiments, at step 2709, each piece of information (i) that the user has access to and (ii) that did not exceed the threshold length and each summary message generated in the step 2708 are arranged into a note stream in chronological or reverse chronological order for display to the user via the note stream interface 2600.

The embodiments described herein have been described with reference to drawings. The drawings illustrate certain details of specific embodiments that implement the systems, methods and programs described herein. However, describing the embodiments with drawings should not be construed as imposing on the disclosure any limitations that may be present in the drawings.

It should be understood that no claim element herein is to be construed under the provisions of 35 U.S.C. § 112(f), unless the element is expressly recited using the phrase “means for.”

As used herein, the term “circuit” may include hardware structured to execute the functions described herein. In some embodiments, each respective “circuit” may include software for configuring the hardware to execute the functions described herein. The circuit may be embodied as one or more circuitry components including, but not limited to, processing circuitry, network interfaces, peripheral devices, input devices, output devices, sensors, etc. In some embodiments, a circuit may take the form of one or more analog circuits, electronic circuits (e.g., integrated circuits (IC), discrete circuits, system on a chip (SOC) circuits), telecommunication circuits, hybrid circuits, and any other type of “circuit.” In this regard, the “circuit” may include any type of component for accomplishing or facilitating achievement of the operations described herein. For example, a circuit as described herein may include one or more transistors, logic gates (e.g., NAND, AND, NOR, OR, XOR, NOT, XNOR), resistors, multiplexers, registers, capacitors, inductors, diodes, wiring, and so on.

Accordingly, the “circuit” may also include one or more processors communicatively coupled to one or more memory or memory devices. In this regard, the one or more processors may execute instructions stored in the memory or may execute instructions otherwise accessible to the one or more processors. In some embodiments, the one or more processors may be embodied in various ways. The one or more processors may be constructed in a manner sufficient to perform at least the operations described herein. In some embodiments, the one or more processors may be shared by multiple circuits (e.g., circuit A and circuit B may comprise or otherwise share the same processor which, in some example embodiments, may execute instructions stored, or otherwise accessed, via different areas of memory). Alternatively or additionally, the one or more processors may be structured to perform or otherwise execute certain operations independent of one or more co-processors. In other example embodiments, two or more processors may be coupled via a bus to enable independent, parallel, pipelined, or multi-threaded instruction execution. Each processor may be implemented as one or more general-purpose processors, application specific integrated circuits (ASICs), field programmable gate arrays (FPGAs), digital signal processors (DSPs), or other suitable electronic data processing components structured to execute instructions provided by memory. The one or more processors may take the form of a single core processor, multi-core processor (e.g., a dual core processor, triple core processor, quad core processor), microprocessor, etc. In some embodiments, the one or more processors may be external to the apparatus, for example the one or more processors may be a remote processor (e.g., a cloud based processor). Alternatively or additionally, the one or more processors may be internal and/or local to the apparatus. In this regard, a given circuit or components thereof may be disposed locally (e.g., as part of a local server, a local computing system) or remotely (e.g., as part of a remote server such as a cloud based server). To that end, a “circuit” as described herein may include components that are distributed across one or more locations.

An exemplary system for implementing the overall system or portions of the embodiments might include a general purpose computing devices in the form of computers, including a processing unit, a system memory, and a system bus that couples various system components including the system memory to the processing unit. Each memory device may include non-transient volatile storage media, non-volatile storage media, non-transitory storage media (e.g., one or more volatile and/or non-volatile memories), etc. In some embodiments, the non-volatile media may take the form of ROM, flash memory (e.g., flash memory such as NAND, 3D NAND, NOR, 3D NOR), EEPROM, MRAM, magnetic storage, hard discs, optical discs, etc. In other embodiments, the volatile storage media may take the form of RAM, TRAM, ZRAM, etc. Combinations of the above are also included within the scope of machine-readable media. In this regard, machine-executable instructions comprise, for example, instructions and data which cause a general purpose computer, special purpose computer, or special purpose processing machines to perform a certain function or group of functions. Each respective memory device may be operable to maintain or otherwise store information relating to the operations performed by one or more associated circuits, including processor instructions and related data (e.g., database components, object code components, script components), in accordance with the example embodiments described herein.

It should also be noted that the term “input devices,” as described herein, may include any type of input device including, but not limited to, a keyboard, a keypad, a mouse, joystick or other input devices performing a similar function. Comparatively, the term “output device,” as described herein, may include any type of output device including, but not limited to, a computer monitor, printer, facsimile machine, or other output devices performing a similar function.

It should be noted that although the diagrams herein may show a specific order and composition of method steps, it is understood that the order of these steps may differ from what is depicted. For example, two or more steps may be performed concurrently or with partial concurrence. Also, some method steps that are performed as discrete steps may be combined, steps being performed as a combined step may be separated into discrete steps, the sequence of certain processes may be reversed or otherwise varied, and the nature or number of discrete processes may be altered or varied. The order or sequence of any element or apparatus may be varied or substituted according to alternative embodiments. Accordingly, all such modifications are intended to be included within the scope of the present disclosure as defined in the appended claims. Such variations will depend on the machine-readable media and hardware systems chosen and on designer choice. It is understood that all such variations are within the scope of the disclosure. Likewise, software and web implementations of the present disclosure could be accomplished with standard programming techniques with rule-based logic and other logic to accomplish the various database searching steps, correlation steps, comparison steps and decision steps.

The foregoing description of embodiments has been presented for purposes of illustration and description. It is not intended to be exhaustive or to limit the disclosure to the precise form disclosed, and modifications and variations are possible in light of the above teachings or may be acquired from this disclosure. The embodiments were chosen and described in order to explain the principals of the disclosure and its practical application to enable one skilled in the art to utilize the various embodiments and with various modifications as are suited to the particular use contemplated. Other substitutions, modifications, changes and omissions may be made in the design, operating conditions and embodiment of the embodiments without departing from the scope of the present disclosure as expressed in the appended claims.

1. A method comprising:
receiving, by one or more processing circuits, a request to provision a plurality of entries associated with a patient;
parsing, by the one or more processing circuits, a database to retrieve the plurality of entries in response to the request;
identifying, by the one or more processing circuits, a source of the request;
determining by the one or more processing circuits, a permission associated with the source of the request;
parsing, by the one or more processing circuits, the plurality of entries based on the permission to retrieve one or more permitted entries of the plurality of entries that are accessible based on the permission; and
displaying, by the one or more processing circuits, a note stream graphical user interface including a note stream providing the one or more permitted entries.

2. The method of claim 1, further comprising arranging, by the one or more processing circuits, the one or more permitted entries in chronological or reverse chronological order based on a timestamp associated with each entry.

3. The method of claim 2, further comprising generating, by the one or more processing circuits, a read receipt associated with a respective entry of the one or more permitted entries in response to the respective entry having been viewed.

4. The method of claim 3, further comprising permitting, by the one or more processing circuits, submission of a new entry by a user in response to a determination that the read receipt associated with the respective entry preceding the new entry has been generated.

5. The method of claim 3, further comprising permitting, by the one or more processing circuits, a selected entry to be viewed in response to a determination that the read receipt associated with the respective entry preceding the selected entry has been generated.

6. The method of claim 5, wherein each entry of the one or more permitted entries provided by the note stream graphical user interface includes a title and the timestamp.

7. The method of claim 6, further comprising expanding, by the one or more processing circuits, the selected entry in response to selection thereof.

8. The method of claim 7, wherein each entry of the one or more permitted entries provided by the note stream graphical user interface includes an expansion key, wherein selection of the expansion key facilitates expanding and collapsing the selected entry.

9. The method of claim 7, wherein, when expanded, the selected entry provides additional information including at least one of a note narrative, a form for completion by the patient, a completed form, test results associated with the patient, prescribed medication details, a patient mood indicator, a patient psychodynamic indicator, or an authorship or creator indicator.

10. The method of claim 9, wherein the additional information includes the note narrative, the patient mood indicator, the patient psychodynamic indicator, and the authorship or creator indicator.

11. The method of claim 1, further comprising:
monitoring, by the one or more processing circuits, a position of a device associated with the patient; and
generating, by the one or more processing circuits, an alert in response to the position of the device indicating that the patient has been at a certain location for more than a threshold period of time.

12. The method of claim 1, further comprising receiving, by the one or more processing circuits, submission of a new entry by a user, wherein the user is one of the patient, a medical professional associated with a treatment of the patient, or a third party associated with the patient but not associated with the treatment of the patient.

13. The method of claim 12, wherein the new entry includes a title populated by the user, further comprising:
comparing, by the one or more processing circuits, a length of the title to a threshold; and
in response to determining that the length exceeds the threshold, at least one of:
(a) providing, by the one or more processing circuits, a notification to the user that the length exceeds the threshold; or
(b) automatically reworking, by the one or more processing circuits, the title to comply with the threshold.

14. The method of claim 12, further comprising:
providing, by the one or more processing circuits, an entry graphical user interface that facilitates the submission of the new entry by the user, wherein the user is the patient, and wherein the entry graphical user interface provides an option to share or not to share the new entry through the note stream; and
preventing, by the one or more processing circuits, the new entry from being included in the note stream or from being selectable within the note stream of a second user that is not the patient even though the second user would otherwise have the permission to view the new entry.

15. A method comprising:
storing, by one or more processing circuits, a plurality of entries associated with a patient in a database;
displaying, by the one or more processing circuits, a note stream graphical user interface with a note stream including at least a subset of the plurality of entries, wherein the subset of the plurality of entries is determined based on a permission of a user accessing the note stream graphical user interface;
arranging, by the one or more processing circuits, the subset of the plurality of entries in an order based on a timestamp associated with each entry;
receiving, by the one or more processing circuits, a selection of a first entry of the subset of the plurality of entries by the user; and
expanding, by the one or more processing circuits, the first entry into an expanded entry in response to a determination that a read receipt associated with a second entry preceding the first entry has been generated, wherein the expanded entry includes additional information that is not visible prior to expansion of the first entry.

16. The method of claim 15, wherein the additional information includes at least one of a note narrative, a form for completion by the patient, a completed form, test results associated with the patient, prescribed medication details, a patient mood indicator, a patient psychodynamic indicator, or an authorship or creator indicator.

17. The method of claim 16, wherein the additional information includes the note narrative, the patient mood indicator, the patient psychodynamic indicator, and the authorship or creator indicator.

18. The method of claim 15, further comprising permitting, by the one or more processing circuits, submission of a new entry to the plurality of entries associated with the patient by the user in response to a determination that read receipts associated with each entry of the subset of the plurality of entries have been generated.

19. The method of claim 15, wherein the user is not the patient, further comprising:
providing, by the one or more processing circuits, an entry graphical user interface that facilitates submission of a new entry by the patient, wherein the entry graphical user interface provides an option to share or not to share the new entry through the note stream; and
preventing, by the one or more processing circuits, the new entry from being included in the subset of the plurality of entries of the note stream or from being selectable within the note stream of the user even though the user would otherwise have permission to view the new entry.

20. A non-transitory computer-readable medium having computer-executable instructions encoded therein, the instructions when executed by one or more processors cause the one or more processors to:
store a plurality of entries associated with a patient in a database;
display a note stream graphical user interface with a note stream including at least a subset of the plurality of entries, wherein the subset of the plurality of entries is determined based on a permission of a user accessing the note stream graphical user interface;
arrange the subset of the plurality of entries in an order based on a timestamp associated with each entry;
receive a selection of a first entry of the subset of the plurality of entries by the user; and
expand the first entry into an expanded entry in response to a determination that a read receipt associated with a second entry preceding the first entry has been generated, wherein the expanded entry includes additional information that is not visible prior to expansion of the first entry.
